(19) United States
(12) Patent Application Publication
(10) Pub. No.: US 2022/0125529 A1
(43) Pub. Date: Apr. 28, 2022

(54) SURGICAL ROBOTIC SYSTEMS

(71) Applicant: Covidien LP, Mansfield, MA (US)

(72) Inventors: Jaimeen Kapadia, Cambridge, MA (US); Ranjan Mishra, Orange, CT (US)

(21) Appl. No.: 17/429,406

(22) PCT Filed: Feb. 12, 2020
(86) PCT No.: PCT/US2020/017865

§ 371 Date: Aug. 9, 2021

Related U.S. Application Data
(60) Provisional application No. 62/806,302, filed on Feb. 15, 2019.

(51) Int. Cl. A61B 34/30; A61B 17/00

(52) U.S. Cl. CPC A61B 34/30; A61B 2017/00477; A61B 2034/305; A61B 17/00

(57) ABSTRACT

An instrument drive unit for use in a robotic surgical system includes a carriage configured to be coupled to a robotic arm, a plurality of drive shafts rotationally supported in the carriage and configured to interface with a corresponding driven member of an electromechanical surgical instrument, a plurality of drive gears fixed to a corresponding drive shaft, a plurality of motors, and a plurality of motor gears operably coupled to a corresponding motor. Each motor gear is configured to rotate a corresponding drive gear in response to an activation of a corresponding motor to actuate a function of the electromechanical surgical instrument.

### BACKGROUND

Surgical robotic systems have been used in minimally invasive medical procedures. Some surgical robotic systems included a console supporting a surgical robotic arm and a surgical instrument having at least one end effector (e.g., forceps or a grasping tool) mounted to the robotic arm. The robotic arm provided mechanical power to the surgical instrument for its operation and movement.

Manually-operated surgical instruments often included a handle assembly for actuating the functions of the surgical instrument. However, when using a robotic surgical system, no handle assembly was typically present to actuate the functions of the end effector. Accordingly, to use each unique surgical instrument with a robotic surgical system, an instrument drive unit was used to interface with the selected surgical instrument to drive operations of the surgical instrument.

The instrument drive unit was typically coupled to the robotic arm via a slide. The slide allowed the instrument drive unit and the attached surgical instrument to move along an axis of the slide, providing a means for adjusting the axial position of the end effector of the surgical instrument.

### SUMMARY

In accordance with an aspect of the present disclosure, an instrument drive unit for use in a robotic surgical system is provided and includes a carriage configured to be coupled to a robotic arm, a plurality of drive shafts rotationally supported in the carriage, a plurality of drive gears, a plurality of motors, and a plurality of motor gears. The drive shafts are configured for interfacing with a corresponding driven member of an electromechanical surgical instrument, the drive gears are fixed to a corresponding drive shaft, and the motor gears are operably coupled to a corresponding motor. Each motor gear is configured to rotate a corresponding drive gear in response to an activation of a respective motor to actuate a function of the electromechanical surgical instrument.

In aspects, the instrument drive unit may further include a plurality of ring gears. The ring gears may operably couple a corresponding motor gear with a corresponding drive gear.

In other aspects, the instrument drive unit may further include a plurality of drive belts. Each drive belt may be disposed about a corresponding motor gear and a corresponding ring gear to operably couple the motor gears with the ring gears.

In some aspects, a first ring gear of the plurality of ring gears may have gear teeth on an inner periphery thereof and on an outer periphery thereof. The gear teeth on the inner periphery of the first ring gear may interface with a corresponding drive gear, and the gear teeth on the outer periphery of the first ring gear may interface with a corresponding drive belt.

In another aspect, the ring gears may be vertically stacked.

In further aspects, a first of the ring gears and a first of the drive gears may be operably coupled to one another and aligned along a first plane; and a second of the ring gears and a second of the drive gears may be operably coupled to one another and aligned along a second plane, vertically displaced from the first plane.

The ring gears may be independently rotatable relative to one another.

In another aspect, the drive gears may be vertically and horizontally offset from one another.

In further aspects, the motor gears may be vertically and horizontally offset from one another.

The instrument drive unit may further include a plurality of motor shafts extending from a corresponding motor. The motor gears may be fixed to a corresponding motor shaft.

In another aspect of the present disclosure, an instrument drive unit for use in a robotic surgical system is provided and includes a carriage configured to be coupled to a robotic arm, a plurality of drive shafts rotationally supported in the carriage, a plurality of drive gears, a plurality of motors, a plurality of motor gears, and a plurality of vertically stacked ring gears. The drive shafts are circumferentially spaced from one another and configured for interfacing with a corresponding driven member of an electromechanical surgical instrument. The drive gears are fixed to a corresponding drive shaft and are disposed at a discrete vertical location relative to one another. The motor gears are operably coupled to a corresponding motor and disposed at a discrete vertical location relative to one another. The ring gears are disposed in the carriage and operably couple a corresponding motor gear with a corresponding drive gear, such that each motor gear is configured to rotate a corresponding drive gear in response to an activation of a respective motor to actuate a function of the electromechanical surgical instrument.

In yet another aspect of the present disclosure, a surgical robotic system is provided and includes a robotic arm, an elongated slide coupled to the robotic arm, and an instrument drive. The instrument drive unit includes a carriage configured to be coupled to the slide, a plurality of drive shafts rotationally supported in the carriage, a plurality of drive gears, a plurality of motors, and a plurality of motor gears. The drive shafts are configured for interfacing with a corresponding driven member of an electromechanical surgical instrument, the drive gears are fixed to a corresponding drive shaft, and the motor gears are operably coupled to a corresponding motor. The motor gears are configured to rotate a corresponding drive gear in response to an activation of a respective motor to actuate a function of the electromechanical surgical instrument.

In aspects, the instrument drive unit may further include a plurality of vertically stacked ring gears. The ring gears may operably couple a corresponding motor gear with a corresponding drive gear. Each ring gear may operably couple a corresponding motor gear with a corresponding drive gear, such that each motor gear is configured to rotate a corresponding drive gear in response to an activation of a respective motor to actuate a function of the electromechanical surgical instrument.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

As used herein, the term “vertical” is understood to be a direction that is parallel to a longitudinal axis of the instrument drive unit. The term “horizontal” is understood to be a direction that is perpendicular to the longitudinal axis of the instrument drive unit.

### BRIEF DESCRIPTION OF THE DRAWINGS

Embodiments of the present disclosure are described herein with reference to the accompanying drawings, wherein:

FIG. 1 is a schematic illustration of a surgical robotic system including an instrument drive unit coupled to a slide in accordance with the present disclosure;

FIG. 2 is a perspective view of the instrument drive unit of the surgical robotic system of FIG. 1 with parts of a carriage of the instrument drive unit shown in phantom, illustrating internal components of the instrument drive unit;

FIG. 3 is a top, perspective view of the internal components of the instrument drive unit of FIG. 2; and

FIG. 4 is a front view of the internal components of the instrument drive unit of FIG. 3.

### DETAILED DESCRIPTION

Embodiments of the presently disclosed surgical robotic system and instrument drive units thereof are described in detail with reference to the drawings, in which like reference numerals designate identical or corresponding elements in each of the several views. As used herein, the term “distal” refers to that portion of the surgical robotic system or component thereof that is closest to the patient, while the term “proximal” refers to that portion of the surgical robotic system or component thereof further from the patient.

As will be described in detail below, provided is an instrument drive unit of a surgical robotic system configured to allow for a bottom-loading of a surgical instrument. The instrument drive unit has a plurality of drive shafts each configured to be coupled to a corresponding driven member of the surgical instrument for carrying out a discrete function of the surgical instrument. The drive shafts of the instrument drive unit are operably coupled to a discrete motor of the instrument drive unit via a plurality of gears and drive belts. The configuration of the gears and drive belts allows for a reduction in the overall height of the instrument drive unit (e.g., the instrument drive unit is more compact). Other features and benefits of the disclosed instrument drive units are further detailed below.

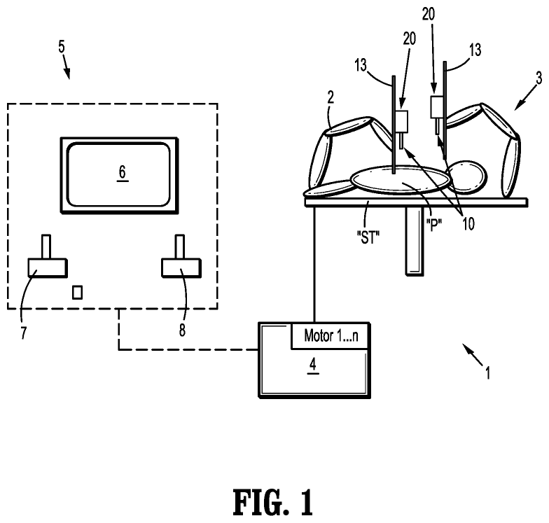

Referring initially to FIG. 1, a surgical system, such as, for example, a surgical robotic system 1, generally includes a plurality of surgical robotic arms 2, 3; an elongated slide 13 coupled to an end of each of the robotic arms 2, 3; an instrument drive unit 20 and an electromechanical instrument 10 removably attached to the slide 13 and configured to move along the slide 13; a control device 4; and an operating console 5 coupled with control device 4. The operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

Each of the robotic arms 2, 3 may be composed of a plurality of members, which are connected through joints. Robotic arms 2, 3 may be driven by electric drives (not shown) that are connected to control device 4. Control device 4 (e.g., a computer) is set up to activate the drives, in particular by means of a computer program, in such a way that robotic arms 2, 3, the attached instrument drive units 20, and thus electromechanical instrument 10 execute a desired movement according to a movement defined by means of manual input devices 7, 8. Control device 4 may also be set up in such a way that it regulates the movement of the instrument drive unit 20 along the slide 13, movement of the robotic arms 2, 3, and/or movement of the drives.

Surgical robotic system 1 is configured for use on a patient “P” lying on a surgical table “ST” to be treated in a minimally invasive manner by means of a surgical instrument, e.g., electromechanical instrument 10. Surgical robotic system 1 may also include more than two robotic arms 2, 3, the additional robotic arms likewise being connected to control device 4 and being telemanipulatable by means of operating console 5. A surgical instrument, for example, an electromechanical surgical instrument 10 (including an electromechanical end effector), may also be attached to the additional robotic arm.

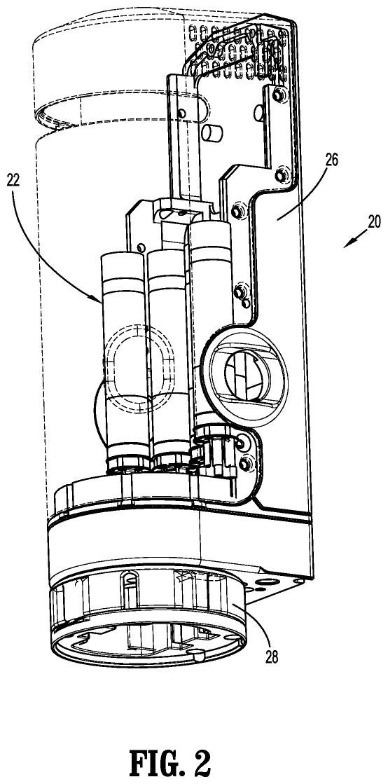

Control device 4 may control a plurality of motors, e.g., motors (Motor 1 . . . n), with each motor configured to drive movement of robotic arms 2, 3 in a plurality of directions. Further, control device 4 may control a plurality of drive motors 22 (FIG. 2) of the instrument drive unit 20 to drive various operations of the surgical instrument 10. The instrument drive unit 20 transfers power and actuation forces from its motors to driven members (not shown) of the electromechanical instrument 10 to ultimately drive movement of components of the end effector of the electromechanical instrument 10, for example, a movement of a knife blade (not shown) and/or a closing and opening of j aw members of the end effector.

For a detailed description of the construction and operation of a robotic surgical system, reference may be made to U.S. Pat. No. 8,828,023, entitled “Medical Workstation,” the entire contents of which are incorporated by reference herein.

With reference to FIGS. 2-4, the instrument drive unit 20 will now be described in detail. The instrument drive unit 20 includes a carriage 26 and a coupling or sleeve 28 rotatably coupled to a distal end portion of the carriage 26 for connecting a surgical instrument 10 (FIG. 1) to the instrument drive unit 20. The carriage 26 of the instrument drive unit 20 is configured to be slidably coupled to a linear track (not shown) defined longitudinally along the slide 13 (FIG. 1). The carriage 26 houses a plurality of drive motors 22a, 22b, 22c, 22d, 22d, 22e (collectively referred herein as “22”) for carrying out various functions of an attached surgical instrument. The sleeve 28 is configured to non-rotationally retain the main body portion of the surgical instrument 10 therein. Accordingly, when the surgical instrument 10 is coupled to the instrument drive unit 20, a rotation of the sleeve 28 results in a rotation of the attached surgical instrument 28.

The motors 22 of the instrument drive unit 20 are concealed within the carriage 26. The drive motors 22 are circumferentially spaced from one another and are independently actuatable via the control device 4 (FIG. 1). One of the drive motors, such as, for example, drive motor 22e, is configured to effectuate a rotation of the surgical instrument 10 when the surgical instrument 10 is coupled to the instrument drive unit 20, and the remaining drive motors 22a, 22b, 22c, 22d are configured to actuate functions of the surgical instrument 10. The drive motors 22 may be cylindrical or pancake motors. Other types of motors are also contemplated. While the instrument drive unit 20 is illustrated as having five drive motors, it is contemplated that the instrument drive unit 20 may have more or less than five drive motors.

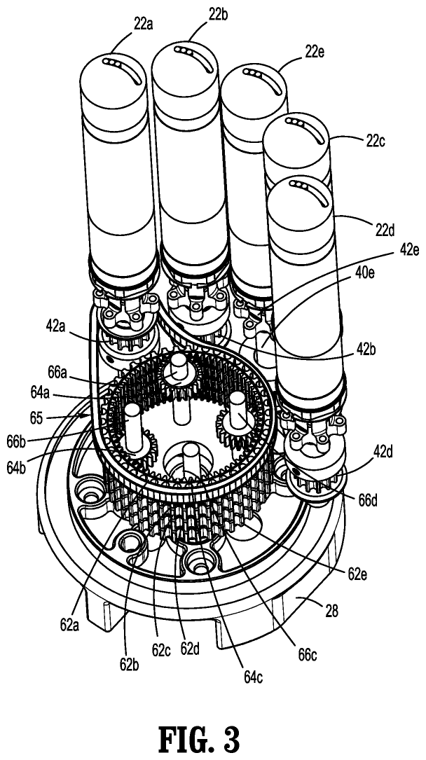

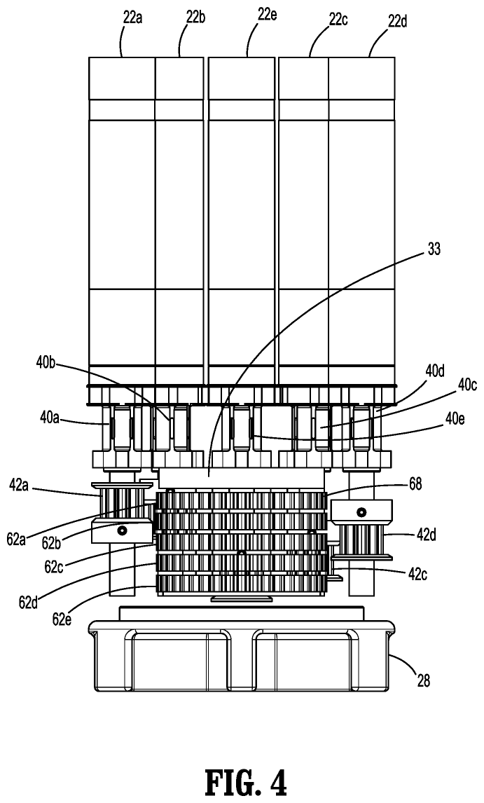

With reference to FIGS. 3 and 4, four drive motors 22a, 22b, 22c, 22d each have a rotatable motor shaft 40a, 40b, 40c, 40d, 40e (collectively referred to herein as “40”) extending distally therefrom. The motor shafts 40 are circumferentially spaced from one another and each has a motor gear 42a, 42b, 42c, 42d, 42e (collectively referred to herein as “42”), such as, for example, a spur gear, rotationally fixed thereabout. Each of the motor gears 42 are positioned at a discrete vertical location on their respective motor shaft 40, such that the motor gears 42 are vertically offset a selected distance from one another. Since the motor gears 42, in addition to be vertically offset from one another, are also circumferentially spaced from one another, the motor gears 42 are offset from one another in all three dimensions.

The instrument drive unit 20 further includes a plurality of ring gears 62a, 62b, 62c, 62d, 62e (collectively referred to herein as “62”). Each of the ring gears 62 has gear teeth 68 extending from both an inner periphery thereof and an outer periphery thereof. The gear teeth 68 on the outer periphery of each of the ring gears 62 interfaces with a corresponding drive belt 65 and the gear teeth 68 on the inner periphery of each of the ring gears 62 interfaces with a corresponding drive gear of a plurality of drive gears 64a, 64b, 64c, 64d (collectively referred to herein as “64”), as will be described. In embodiments, each of the rings gears 62 may be constructed from inner and outer ring gears integrally formed with one another. While only one drive belt 65 is illustrated, it is contemplated that each ring gear 62 and motor gear 42 has a corresponding drive belt disposed thereabout. The drive belt 65 may be flexible or rigid. In embodiments, instead of having teeth, the drive belt 65 may frictionally engage the outer periphery of the ring gears 62 and the motor gears 42.

The instrument drive unit 20 further includes a plurality of drive shafts 66a, 66b, 66c, 66d (collectively referred to herein as “66”). A distal end portion of each of the drive shafts 66 is configured to operably couple to a driven member (not explicitly shown) of the surgical instrument 10. For example, the distal end portion of each of the drive shafts 66 may have a coupler (e.g., a gear) for coupling with a corresponding coupler of a driven member of the surgical instrument 10. Accordingly, upon bottom-loading of the electromechanical instrument 10 into the instrument drive unit 20, the distal end portions of the drive shafts 66 of the instrument drive unit 20 operably couple to the gears/couplers in a distal end of the main body portion (not shown) of the electromechanical instrument 10, such that a rotation of each drive shaft 66 rotates a correspondingly coupled driven member of the surgical instrument 10 to effectuate a discrete function of the surgical instrument (e.g., opening/closing of the end effector, articulation of the end effector, etc.)

The drive shafts 66 each have a drive gear 64 such as, for example, a spur gear, rotationally fixed thereabout. Each of the drive gears 64 are positioned at a discrete vertical location on their respective drive shaft 66, such that the drive gears 64 are vertically offset a selected distance from one another. Since the drive gears 64, in addition to being vertically offset, are also circumferentially spaced from one another, the drive gears 64 are offset from one another in all three dimensions. As mentioned above, the drive gears 64 each interface or intermesh with the gear teeth 68 on the inner periphery of a corresponding ring gear 62 and receive torque therefrom originating from the respective motor 22.

The instrument drive unit 20 may include a slip ring 33 (FIG. 4) received disposed adjacent the proximal-most ring gear 62a. The slip ring 33 transfers electrical signals or power between fixed structures (e.g., the drive motors 22) and rotating structures (e.g., the electromechanical surgical instrument 10). The electrical signals transferred by the slip ring 33 may be feedback signals from the electromechanical surgical instrument 10 relating to the status and location of the surgical instrument 10 and/or the status and location of adjacent tissue structures. For example, the feedback may include the temperature of the surgical instrument 10, forces experienced by the surgical instrument 10, and/or the position of certain structures of the surgical instrument 10 relative to one another or relative to the adjacent tissue structures.

Further details regarding the instrument drive unit 20 may be found in U.S. Provisional Application No. 62/693,488, filed on Jul. 3, 2018, the entire contents of which being incorporated by reference herein.

In operation, the electromechanical instrument 10 is coupled to the instrument drive unit 20 by passing the main body portion of the electromechanical instrument 10 through the sleeve 28 of the instrument drive unit 20 in a proximal direction. With the main body portion of the electromechanical instrument 10 attached to the sleeve 28 of the instrument drive unit 28, the distal end portion of each of the drive shafts 66 interfaces with corresponding gears/couplers (not shown) in the proximal end of the main body portion of the electromechanical instrument 10.

To actuate a particular function of the surgical instrument 10, such as, for example, an opening or closing of an end effector of the surgical instrument 10, one of the drive motors 22 of the instrument drive unit 20, such as the first drive motor 22a, is activated via the control device 4 (FIG. 1). An activation of the first drive motor 22a rotates the first motor shaft 40a. Rotation of the first motor shaft 40a transfers torque from the first motor shaft 40a to a first driven member of the electromechanical instrument 10.

In particular, the first motor gear 42a rotates with the first motor shaft 40a, which, in turn, rotates the first drive belt 65. Rotation of the first drive belt 65 drives a rotation of the first ring gear 62a, which, in turn, rotates the first drive gear 64a. Since the first drive gear 64a is rotationally fixed about the first drive shaft 66a, and the distal end portion of the first drive shaft 66a is operably coupled to the proximal end of the first driven member of the surgical instrument 10 (FIG. 1), a rotation of the first drive gear 64a causes the first drive shaft 66a to rotate, thereby rotating the first driven member of the electromechanical instrument 10 to actuate an associated function of the surgical instrument 10. The drive motor 22e may be configured to resist rotation of the motor shaft 40e thereof during actuation of any of the drive motors 22a, 22b, 22c, 22d so that actuation of one of the drive motors 22a, 22b, 22c, 22d does not inadvertently result in a rotation of the sleeve 28.

To rotate the electromechanical instrument 10 about its longitudinal axis, the fifth drive motor 22e of the instrument drive unit 20 is activated by the control device 4 (FIG. 1). As noted above, an activation of the fifth drive motor 22e rotates the sleeve 28. Given that the electromechanical instrument 10 is non-rotationally supported in the sleeve 28, the electromechanical instrument 10 rotates with the sleeve 28 relative to the carriage 26 to change a rotational orientation of the electromechanical instrument 10. The drive motors 22a, 22b, 22c, 22d may be configured to concurrently rotate the motor shafts 40a, 40b, 40c, 40d, and in turn the drive gears 64a, 64b, 64c, 64d, with the sleeve 28 rotation. This would prevent rotation of the drive shafts 66a, 66b, 66c, 66d relative to the ring gears 62a, 62b, 62c, 62d during rotation of the sleeve 28, which may otherwise occur if the drive gears 64a, 64b, 64c, 64d were allowed to rotate relative to the ring gears 62a, 62b, 62c, 62d during rotation of the sleeve 28.

As can be appreciated, the instrument drive unit 20 described above improves usability of the surgical robotic system 1, reduces a foot-print of the overall system 1, improves safety architecture, reduces the time required to remove surgical instruments in case of an emergency, and simplifies the electronics used in the instrument drive unit 20.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. An instrument drive unit for use in a robotic surgical system, the instrument drive unit comprising:
a carriage configured to be coupled to a robotic arm;
a plurality of drive shafts rotationally supported in the carriage, the plurality of drive shafts configured for interfacing with a corresponding driven member of an electromechanical surgical instrument;
a plurality of drive gears, each drive gear of the plurality of drive gears fixed to a corresponding drive shaft of the plurality of drive shafts;
a plurality of motors; and
a plurality of motor gears, each motor gear of the plurality of motor gears operably coupled to a corresponding motor of the plurality of motors, wherein each motor gear of the plurality of motor gears is configured to rotate a corresponding drive gear of the plurality of drive gears in response to an activation of a respective motor of the plurality of motors to actuate a function of the electromechanical surgical instrument.

2. The instrument drive unit according to claim 1, further comprising a plurality of ring gears, each ring gear of the plurality of ring gears operably coupling a corresponding motor gear of the plurality of motor gears with a corresponding drive gear of the plurality of drive gears.

3. The instrument drive unit according to claim 2, further comprising a plurality of drive belts, each drive belt of the plurality of drive belts disposed about a corresponding motor gear of the plurality of motor gears and a corresponding ring gear of the plurality of ring gears to operably couple the motor gears with the ring gears.

4. The instrument drive unit according to claim 3, wherein at least a first ring gear of the plurality of ring gears has gear teeth on an inner periphery thereof and on an outer periphery thereof, wherein the gear teeth on the inner periphery of the first ring gear interface with a corresponding drive gear of the plurality of drive gears, and the gear teeth on the outer periphery of the first ring gear interface with a corresponding drive belt of the plurality of drive belts.

5. The instrument drive unit according to claim 2, wherein the plurality of ring gears are vertically stacked.

6. The instrument drive unit according to claim 5, wherein a first ring gear of the plurality of ring gears and a first drive gear of the plurality of drive gears are operably coupled to one another and aligned along a first plane, and wherein a second ring gear of the plurality of ring gears and a second drive gear of the plurality of drive gears are operably coupled to one another and aligned along a second plane, vertically displaced from the first plane.

7. The instrument drive unit according to claim 2, wherein the plurality of ring gears are independently rotatable relative to one another.

8. The instrument drive unit according to claim 1, wherein the plurality of drive gears are vertically and horizontally offset from one another.

9. The instrument drive unit according to claim 1, wherein the plurality of motor gears are vertically and horizontally offset from one another.

10. The instrument drive unit according to claim 1, further comprising a plurality of motor shafts, each motor shaft of the plurality of motor shafts extending from a corresponding motor of the plurality of motors, each motor gear of the plurality of motor gears being fixed to a corresponding motor shaft of the plurality of motor shafts.

11. An instrument drive unit for use in a robotic surgical system, the instrument drive unit comprising:
a carriage configured to be coupled to a robotic arm;
a plurality of motors supported in the carriage;
a plurality of motor shafts, each motor shaft of the plurality of motor shafts extending distally from a corresponding motor of the plurality of motors, each motor shaft of the plurality of motor shafts having a motor gear fixed thereabout; and
a plurality of drive shafts circumferentially spaced from one another and configured for interfacing with a corresponding driven member of an electromechanical surgical instrument, each drive shaft of the plurality of drive shafts having a drive gear fixed thereabout, each drive gear being disposed at a discrete vertical location relative to one another, wherein each motor gear is configured to rotate a corresponding drive gear in response to an activation of a respective motor of the plurality of motors to actuate a function of the electromechanical surgical instrument.

12. The instrument drive unit according to claim 11, further comprising a plurality of vertically stacked ring gears, each ring gear of the plurality of ring gears operably coupling a corresponding motor gear with a corresponding drive gear, such that each motor gear is configured to rotate a corresponding drive gear in response to an activation of a respective motor of the plurality of motors to actuate a function of the electromechanical surgical instrument.

13. The instrument drive unit according to claim 12, further comprising a plurality of drive belts, each drive belt of the plurality of drive belts disposed about a corresponding motor gear and a corresponding ring gear of the plurality of ring gears to operably couple the motor gears with the ring gears.

14. The instrument drive unit according to claim 13, wherein at least a first ring gear of the plurality of ring gears has gear teeth on an inner periphery thereof and on an outer periphery thereof, wherein the gear teeth on the inner periphery of the first ring gear interface with a corresponding drive gear, and the gear teeth on the outer periphery of the first ring gear interface with a corresponding drive belt of the plurality of drive belts.

15. The instrument drive unit according to claim 12, wherein a first ring gear of the plurality of ring gears and a first drive gear attached to a first drive shaft of the plurality of drive shafts are operably coupled to one another and aligned along a first plane, and wherein a second ring gear of the plurality of ring gears and a second drive gear attached to a second drive shaft of the plurality of drive shafts are operably coupled to one another and aligned along a second plane, vertically displaced from the first plane.
